(19) United States
(12) Patent Application Publication
(10) Pub. No.: US 2001/0047531 A1
(43) Pub. Date: Dec. 6, 2001

(54) Forearm guard

(76) Inventor: Alberto Spies, Yonkers, NY (US)

Correspondence Address: LIEBERMAN & NOWAK LLP, 350 FIFTH AVE., SUITE 7412, NEW YORK, NY, 10118, US

(21) Appl. No.: 09/863,645

(22) Filed: May 23, 2001

Related U.S. Application Data
(60) Provisional application No. 60/208,220, filed on May 30, 2000.

(51) Int. Cl. A41D 13/08; A61F 9/00; A41D 13/06

(52) U.S. Cl. 2/16; 2/22

(57) ABSTRACT

A forearm guard is disclosed wherein the components thereof provides both effective impact protection to the forearm and perspiration absorbing capability to prevent moisture from effecting the user's grip. In the preferred embodiments of the invention, the forearm guard comprise a sleeve of stretchable material and a flexible impact absorbing pad affixed to the forearm portion of said sleeve. The sleeve is preferable comprised of washable fabric that includes an elastomeric component, and/or is woven to provide stretchability. The sleeve can be of any suitable length and diameter effective to secure the impact absorbing pad over the forearm and thereby protect the forearm of the wearer during an athletic activity. In the preferred embodiments of this invention, the forearm guard is particularly suitable in the protection of the forearm of a baseball player from a wild pitch during batting.

### CROSS-REFERENCE TO RELATED APPLICATIONS

This application claims priority to Provisional Application Serial No. 60/208,220 filed on May 30, 2000.

### BACKGROUND OF THE INVENTION

1. Field of the Invention

This invention relates to an article of protective sports gear for protection of the forearm of a baseball player during batting.

2. Description of the Prior Art

The utilization of protective gear in both contact and non-contact sports is well-known. In baseball, the use of protective gear was initially the exclusive province of the catcher, and the home plate umpire. Gradually, the ballplayers adopted protective head gear, shin guards and, more recently, guards to protect their forearm while batting.

The following patents are representative of the state of the art in protective gear used to protect the forearms of individuals.

U.S. Pat. No. 4,707,861 (to Lavoie, issued Nov. 24, 1987) discloses a forearm shield pad which can be releasably secured to a forearm of a person by surrounding straps. The pad includes a sturdy, rigid core and a shock-dampening surrounding sheath. The Lavoie device can best be described as a type of body armor for use by law enforcement and correctional officers in their physical confrontations with aggressive individuals. Accordingly, the exterior face of the pad also includes a longitudinal channel member into which may be releasably engaged a cylindrical striking club of the prison warden.

U.S. Des. Pat. No. 300,676 (to Pierce, issued Apr. 11, 1989) discloses a unique and ornamental design for a forearm guard which comprises a fabric sleeve and a series of protective (impact absorbing) pads affixed to the sleeve. In the embodiment of the invention illustrated in the Pierce design, the impact absorbing pads appear as distinct series of rigid, articulating segments arranged along a sleeve. The segmentation of the impact absorbing pads is calculated to permit flexure of the forearm guard and, thus, unrestrained freedom or movement/articulation of the forearm.

Notwithstanding the protection purportedly offered by each of the foregoing devices, additional improvement is necessary to provide protection, that is both light weight, and readily adaptable to a sports environment (e.g. baseball), while at the same time preserving freedom of movement without discomfort or distraction.

### OBJECTS OF THE INVENTION

It is the objects of this invention to remedy the above and related deficiencies in the prior art.

More specifically, it is the principle object of this invention to provide a forearm guard suitable for use in a sports environment, such as baseball.

It is another object of this invention to provide a forearm guard suitable for use in a sports environment wherein the guard is both light weight and can be put on and removed without any mechanical fasteners (e.g. Velcro), straps, or other releasable means.

It is yet another object of this invention to provide a forearm guard suitable for use in a baseball batting environment that provides both protection to the forearm and is absorbing of perspiration that can cause the baseball bat to slip from the batter's hands.

Additional objects of this invention include the integration of the forearm guard into sports apparel (e.g., sweat shirts sleeves).

### SUMMARY OF THE INVENTION

The above and related objects are achieved by providing a forearm guard which comprises a sleeve of stretchable material and a flexible impact absorbing pad affixed to the forearm portion of said sleeve. The sleeve is preferable comprised of washable fabric that includes an elastomeric component, and/or is woven to provide stretchability. The sleeve can be of any suitable length and diameter effective to secure the impact absorbing pad over the forearm and thereby protect the forearm of the wearer during an athletic activity. In the preferred embodiments of this invention, the forearm guard is particularly suitable in the protection of the forearm of a baseball player from a wild pitch during batting. Thus, the structure of the forearm guard, as contemplated by this invention, preserves freedom of movement and flexure of the forearm during the swinging of the baseball bat, while at the same time retaining the shock absorbing pad in position to protect the batter from injury. The forearm guard of this invention also includes the additional feature of incorporation of a sweat band-like function into the sleeve, either by the fabrication of the entire sleeve of a terry cloth-like material, or simply including such terry cloth like material at the proximal end of the sleeve adjacent to the batter's wrist.

In another of the preferred embodiments of this invention, the forearm guard can be included/integrated as a component of sports apparel (sweat shirt); and, included on each sleeve thereof

### BRIEF DESCRIPTION OF THE DRAWINGS

FIG. 1 depicts a perspective view of the forearm guard of this invention.

FIG. 2 depicts a schematic of the sleeve of the forearm guard of this invention, including its overall dimensions.

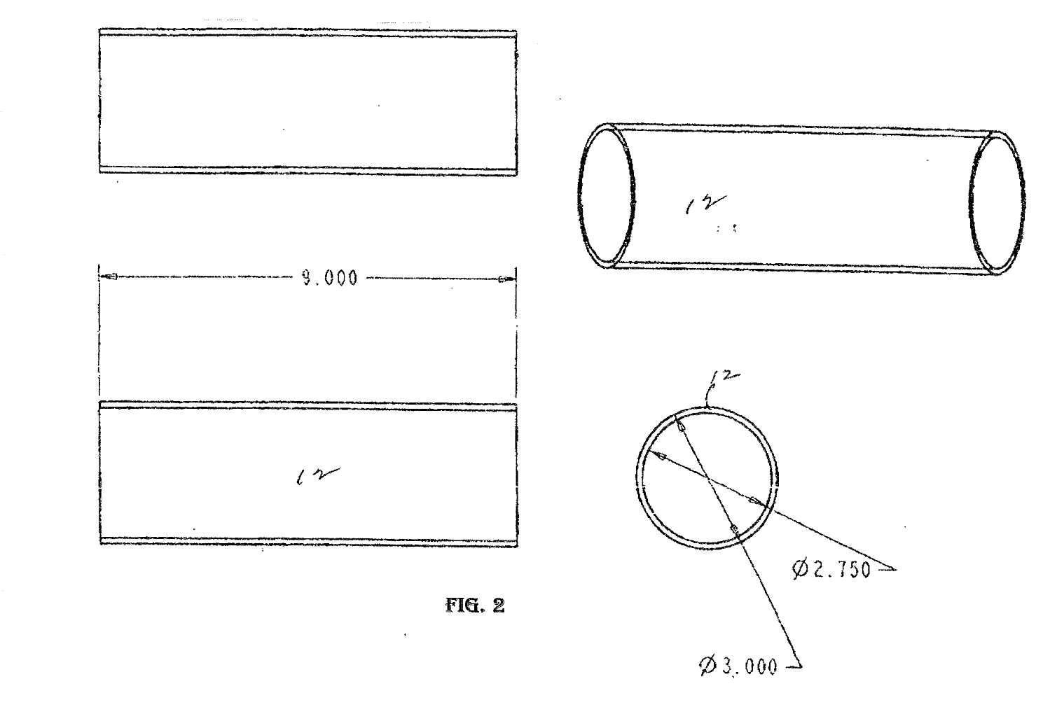

FIG. 3 depicts a schematic of the protective pad of the forearm guard of this invention, including its overall dimensions

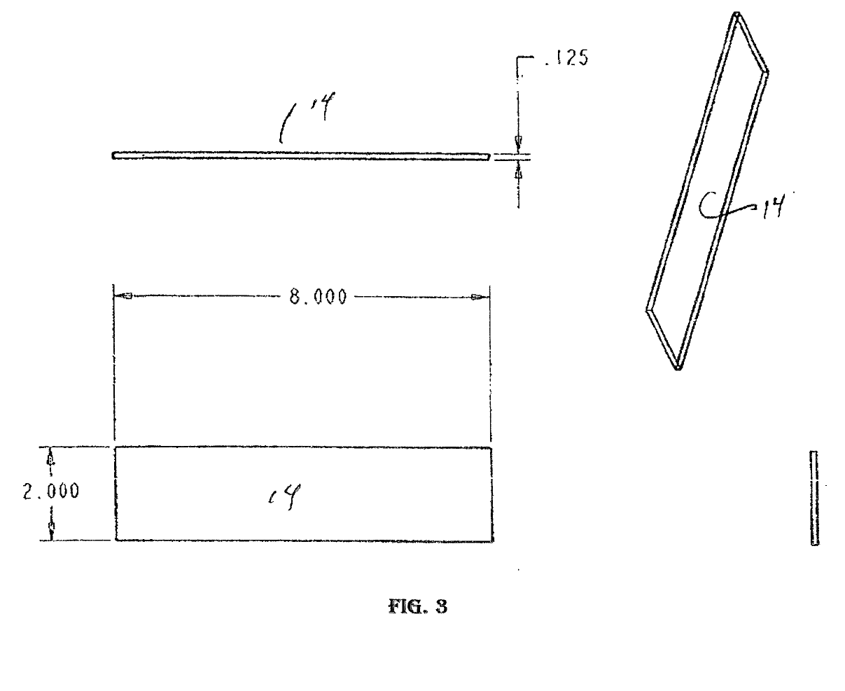

### DETAILED DESCRIPTION OF THE INVENTION INCLUDING PREFERRED EMBODIMENTS

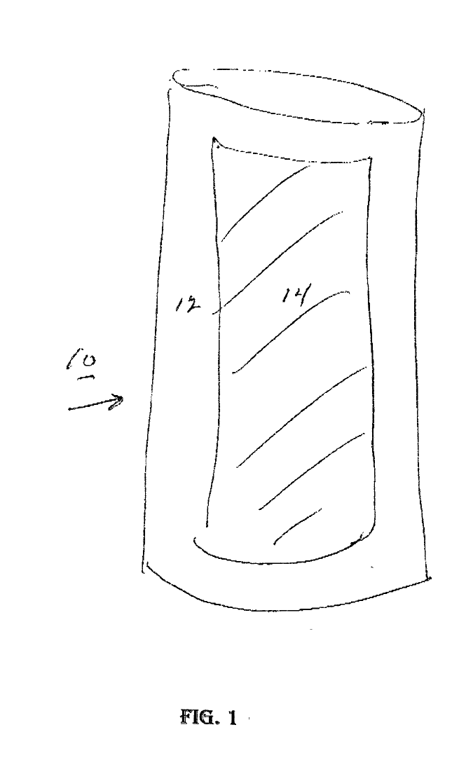

The forearm guard (10) of this invention shown in FIG. 1 is designed for use in the a baseball sports environment, specifically in the protection of the forearm of a baseball player during batting. The guard (10) depicted in FIG. 1 comprises two basic components, specifically, an elastomeric sleeve (12) and a shock/impact absorbing pad (14) associated therewith. Each of these components are distinct and yet associated with one another to provide both effective protection of the forearm during batting and absorption of perspiration from the batter's arm, so as to prevent moisture from loosening his grip on the baseball bat. In the preferred embodiments of this invention, the sleeve is typically tubular in nature and designed to extend from just above the batter's wrist to just below the elbow. The flexible compliant nature of the sleeve, (e.g. a terry cloth fabric incorporation an elastomeric yarn or insert) insures both compliance with the form of the batter's forearm, and resistance to movement along the length of the arm during swing of the bat or arm movements associated with running. Because of the relatively light weight construction of this guard, the elastomeric tension needed to retain the guard in place on the forearm is relative small, thus, insuring maximum freedom of movement of the forearm during athletic activity.

Similarly, the shock absorbing pad, that is associated with the sleeve, is also formed of a relatively flexible impact resistant material (e.g., expandable foam or elastomer). The material of choice for this pad (e.g. neoprene rubber) is relatively flexible at an effective impact absorbing thickness, which is typically less than about 0.25 inches and at least about 0.125 inches in thickness. It is understood that the thickness of the shock absorbing pad is limited by practical considerations relative to flexure of the guard and effectiveness in protection. In the preferred embodiments of this invention, the composite structure resulting from the sleeve and shock absorbing pad achieve the requisite protection without adding substantial weight to the device, or causing movement limiting restriction on the athlete's forearm. In another of the preferred embodiments of this invention the impact resistant pad is either permanently affixed to the sleeve, or alternatively, releasably attached to the sleeve with a Velcro-like fastener.

In another of the preferred embodiment of this invention, the forearm guard is integrated into a garment (e.g., sweat shirt); and, most preferably into both sleeves thereof, to afford maximum protection to the wearer.

While the forearm guard of this invention has been described in reference to baseball, it is understood that such device, particularly when incorporated into a sports shirt, may also be a desirable addition to a team sports uniform (e.g.,, a flag/touch football, or basketball), where limited contact between players often occurs. Additional uses of the forearm guard of this invention may also find application in work closes or comparable protective clothing and uniforms in the workplace.

What is claimed is:

1. A forearm guard having a sleeve and an impact absorbing pad associated therewith, comprising:
A. A sleeve of essentially tubular construction wherein the overall length thereof conforms essentially to the linear dimension of a forearm of a wearer, and the overall diameter thereof conforms essentially to approximately 75% of the diameter of the narrower of the dimension of the forearm of said wearer,
Said sleeve being fabricated from a woven fabric which either by virtue of its intrinsic properties and/or weaving, is essentially resilient and stretchable so as to both conform to the said forearm and retain said guard in place during movement of said forearm,
B. An impact absorbing pad comprising an expandable foam or elastomer, said pad including a effective impact absorbing thickness of said expandable foam or elastomer, and
C. Means for attachment of said impact absorbing pad to said sleeve.

2. The forearm guard of claim 1, wherein said sleeve is fabricated from a moisture absorbent material.

3. The forearm guard of claim 1, wherein said sleeve is fabricated from a terry cloth-like material.

4. The forearm guard of claim 1, wherein said impact resistant pad comprises neoprene.

5. The forearm guard of claim 1, wherein said impact resistant pad comprises neoprene and has a effective impact resistant thickness of from about less than about 0.25 inches and at least about 0.125 inches.

6. The forearm guard of claim 1, wherein the impact resistant pad is releasably attached to said sleeve
